SEQUENCE LISTING (2 sequences)

SEQ ID NO: 1 - DNA, 3280 bp, Homo sapiens
gtcgggggccgaagcgcctcccgcagggagtccccgtgaggccgcgctgccgcgttacccgcagcggccgggggtccggggagtgcggggctccgagacagagccaggcgccccccagcggtcggcgcgggccccatggctgggccgggcggagcggaaccggtgaggtgaagccccggggccggcagccggaggcgcgtggccggggcgccggctccatgggcagcggcacacggcggcgcgatgatggacgttaacagcagcggccgcccggacctctacgggcacctccgctctttccttctgccagaagtggggcgcgggctgcccgacttgagccccgacggtggcgccgacccggtcgcgggctcctgggcgccgcacctgctgagcgaggtgacagccagcccggcgcccacctgggacgcgcccccggacaatgcctccggctgtggggaacagatcaactacggcagagtcgagaaagttgtgatcggctccatcctgacgctcatcacgctgctgacgatcgcgggcaactgcctggtggtgatctccgtgtgcttcgtcaagaagctccgccagccctccaactacctgatcgtgtccctggcgctggccgacctctcggtggctgtggcggtcatgcccttcgtcagcgtcaccgacctcatcgggggcaagtggatctttggacactttttctgtaatgtcttcatcgccatggacgtcatgtgctgcacggcctcgatcatgaccctgtgcgtgatcagcattgacaggtaccttgggatcacaaggcccctcacataccctgtgaggcagaatgggaaatgcatggcgaagatgattctctccgtctggcttctctccgcctccatcaccttacctccactctttggatgggctcagaatgtaaatgatgataaggtgtgcttgatcagccaggactttggctatacgatttactctaccgcagtggcattttatatccccatgtccgtcatgcttttcatgtactaccagatttacaaggctgccaggaagagtgctgccaaacacaagtttcctggcttccctcgagtggagccagacagcgtcatcgccctgaatggcatagtgaagctccagaaggaggtggaagagtgtgcaaacctttcgagactcctcaagcatgaaaggaaaaacatctccatctttaagcgagaacagaaagcagccaccaccctggggatcatcgtcggggcctttaccgtgtgctggctgccatttttcctcctctcgacagccagacccttcatctgtggcacttcctgcagctgcatcccactgtgggtggagaggacatttctgtggctaggctatgcaaactctctcattaacccttttatatatgccttcttcaaccgggacctgaggaccacctatcgcagcctgctccagtgccagtaccggaatatcaaccggaagctctcagctgcaggcatgcatgaagccctgaagcttgctgagaggccagagagacctgagtttgtgctacaaaatgctgactactgtagaaaaaaaggtcatgattcatgattgaaagcagaacaatggagatgaaataaacaaggcaaaatagaggtggaaacagaaggaagtcatttgctgagtctgcagaatggaatgcagcttctgtcctttcttgggatgtctaaaacatgacaaacagggtgatctgttgtacacactatcttatgagggagatggtgacttctccttttttctgtggatcagtgctattgtgtgttctcagtttaagatagcagatcatctcagcagtaagcacaccaacagaactgagttccagaaaggaagcagtttctggtgctttgcatatgtccaaatccatgcaagtgggagaaagttccagtgcacacttcccatgcttctgagtctaggcgttgtggtgaatatgcaggaatcattcatgagacagaatgtattttgttgtatgacagaaggcttttccaagcaaactgcggtgagcgtagtgttgaatatgttgcatgtccatgttagaaaacagaatccagtcatcagctaatacaaatgatttcccagagcagtgtttgtttcaagcttctgtcaaatagtttggcttttctgcagggtctctgtgatttccccacctatgtacttttctgtgttaactcacttattatggtataactcaggaacaggtctcaggatggagagaagcatacaacatgagcaaagctttttctcgtgctctgaagggtcttgagaggggagtgggatgccttctgtgaggatttttatcgcagtgaagctcaagtggtacccaactgcgttgtaattaacatgattattggcatgtttttgactctcattttctacaaggctgcagtgggcccacccatgggctggcccataactgtatgttatgtggaagaagtgtgggaatgatgggttttccatgaaatgtgatgcttctggagatggggcataaacatgttcattcttgttgagctgcattgtgttaaaccaaattgtgtgaagcgaggtcacctggaccactcttgggaaatgcttcagaacaccagtctgttccttctagagaatttgtttttgtgttctatgtatcttaaatcaaatgtgtgcctaagcaattctgtcctctgattcccacccctacccaccaaaaaatggtagacatgaaaggcaggtgaggaaacagaaaaggtctctttgcagatatatattttttaaatggcactatgtagtaattaagagctaaggagacagaaagacagtagatgagaatagaagagtacctgtacctacaaggggaaaaatgaatgcaattctttatttgataagagtgaactgaaagagaatattattttcctacaataaagcaagctgtcctctcacaggataaagtggaattctgacatggattatggacttcttctaactcccattgacattgcttcttaccagacatggggaaaagctacctttcctgaaaggggaggagggcactgtcctttcagacaaagcttgtacaaattcctgaaccgcaaatttgctgaagtgaccgtatatattatattcataatttaaatgattatttatggtcagatacatattgttaaacttgaaaatgttttattacattacatcaaataaagtttatttgtagctgaaataaagcaacgtaagtaacaatttttaattaaaaaaaaaaaaaaaaaaaaaaaaaaaaaaaaaa

SEQ ID NO: 2 - protein, 445 aa, Homo sapiens
MMDVNSSGRPDLYGHLRSFLLPEVGRGLPDLSPDGGADPVAGSWAPHLLSEVTASPAPTWDAPPDNASGCGEQINYGRVEKVVIGSILTLITLLTIAGNCLVVISVCFVKKLRQPSNYLIVSLALADLSVAVAVMPFVSVTDLIGGKWIFGHFFCNVFIAMDVMCCTASIMTLCVISIDRYLGITRPLTYPVRQNGKCMAKMILSVWLLSASITLPPLFGWAQNVNDDKVCLISQDFGYTIYSTAVAFYIPMSVMLFMYYQIYKAARKSAAKHKFPGFPRVEPDSVIALNGIVKLQKEVEECANLSRLLKHERKNISIFKREQKAATTLGIIVGAFTVCWLPFFLLSTARPFICGTSCSCIPLWVERTFLWLGYANSLINPFIYAFFNRDLRTTYRSLLQCQYRNINRKLSAAGMHEALKLAERPERPEFVLQNADYCRKKGHDS

(19) United States
(12) Patent Application Publication
(10) Pub. No.: US 2013/0287705 A1
(43) Pub. Date: Oct. 31, 2013

(54) METHOD OF TREATING MUCOSAL INFLAMMATION

(71) Applicants: Waliul I. Khan, Hamilton (CA); Janice J. Kim, Richmond Hill (CA); Jean-Eric Ghia, Hamilton (CA); Huaqing Wang, Hamilton (CA)

(72) Inventors: Waliul I. Khan, Hamilton (CA); Janice J. Kim, Richmond Hill (CA); Jean-Eric Ghia, Hamilton (CA); Huaqing Wang, Hamilton (CA)

(73) Assignee: MCMASTER UNIVERSITY, Hamilton, ON (CA)

(21) Appl. No.: 13/882,782

(22) PCT Filed: Nov. 3, 2011
(86) PCT No.: PCT/CA11/01239

§ 371 Date: Jul. 15, 2013

Related U.S. Application Data
(60) Provisional application No. 61/409,554, filed on Nov. 3, 2010.

(51) Int. Cl. A61K 31/454

(52) U.S. Cl. CPC A61K 31/454
USPC 424/10.1; 514/326

(57) ABSTRACT

A method of treating mucosal inflammation associated with a pathological condition in a mammal is provided. The method comprises the step of inhibiting 5-HT signaling at a target site in order to block 5-HT7 receptor function.

### FIELD OF THE INVENTION

The present invention generally relates to the treatment of inflammation in the mucosa, and more particularly to a treatment method in which 5-HT signaling is modulated.

### BACKGROUND OF THE INVENTION

The gastrointestinal (GI) tract contains the largest endocrine organ in the body and is made up of an extensive system of endocrine cells. EC cells are the best characterized enteric endocrine cell population which synthesize and release the biogenic amine serotonin (5-hydroxytryptamine; 5-HT). The GI tract contains about 95% of the body's 5-HT and over 90% of this supply is synthesized and stored within EC cells. 5-HT is released from EC cells into the blood, into the surrounding tissue and into the gut lumen and participates in various gut functions, and has been implicated in several GI disorders emphasizing the significance of 5-HT in intestinal homeostasis. Secretion of 5-HT by EC cells can be enhanced or attenuated by the action of signaling molecules released from surrounding cells including immune cells and alteration of 5-HT release may contribute to intestinal physiology and pathophysiology.

Inflammatory Bowel Disease (IBD) includes two chronic gastrointestinal (GI) diseases, ulcerative colitis (UC) and Crohn's disease (CD), which are relapsing inflammatory conditions of unknown etiology. IBD is the most common and serious chronic inflammatory condition of the human bowel. Mucosal changes in IBD are characterized by ulcerative lesions accompanied by a prominent infiltrate of activated cells from both the innate and adaptive immune systems. In addition to immune cells, inflammation in the gut is associated with an alteration in EC cells numbers and 5-HT amount. Changes in intestinal EC cell numbers and 5-HT are observed in patients with IBD and also in experimental colitis. Due to the strategic location of EC cells in gut mucosa, it is very likely that 5-HT plays an important role in immune activation and in generation of gut inflammation including IBD.

EC cells synthesize 5-HT from its precursor L-tryptophan. Tryptophan hydroxylase (TPH) catalyzes the rate-limiting step in the synthesis of 5-HT from tryptophan and has been detected prominently in EC cells. Recent studies have shown that there are two isoforms of TPH enzymes regulating the 5-HT system. TPH1 is mainly present in peripheral organs such as the intestine, while TPH2 predominates in the brain stem. Thus, 5-HT seems to be synthesized independently in peripheral tissues and neurons by two different rate-limiting TPH isoenzymes. Recently, by utilizing tryptophan hydroxylase 1-deficient (TPH1^{-/-}) mice which have a significantly reduced amount of 5-HT in the gut, and mice treated with 5-HT synthesis inhibitor (inhibitor for both TPH1 and TPH2) para-chloro-D, L-phenylalanine (PCPA), a critical role of 5-HT in the generation of colitis in two different models of experimental colitis (dextran sulfate sodium (DSS) and dinitrobenzene sulfonic acid (DNBS)) was demonstrated. Delayed onset, decreased severity of colitis and down-regulation of pro-inflammatory cytokine production were observed in TPH1^{-/-} mice as compared to wild-type mice and in PCPA treated mice after induction of colitis. These results corroborate with the recent studies which demonstrated that chemical-induced colitis by trinitrobenzene sulphonic acid (TNBS) or spontaneous colitis associated with IL-10 deficiency is increased in severity when coupled with the 5-HT-enhancing effects of the knockout of serotonin reuptake transporter (SERT). It has also been demonstrated that dendritic cells isolated from TPH1^{-/-} mice in DSS-colitis produced reduced IL-12 compared to TPH1^{+/+} mice and stimulation with 5-HT restored IL-12 production from the dendritic cells. In addition, there was an up-regulation of severity of inflammation in TPH1^{-/-} mice after adoptive transfer of DCs pulsed with 5-HT.

In recent years significant progress has been made in understanding the pathogenesis of IBD which has led to improved strategies to control inflammation through the use of immunosuppressive drugs and the antibody targeting of tumor necrosis factor (TNF)α. However, treatment using these drugs may cause many side effects such as toxicity in the case of immunosuppressive agents, acute infusion reactions, and the development of antibodies to the anti-TNF-α antibody.

In view of the drawbacks associated with current IBD treatments, it would be desirable to develop a novel treatment protocol for pathological conditions associated with mucosal inflammation.

### SUMMARY OF THE INVENTION

It has now been determined that inhibition of 5-HT signaling by targeting the 5HT7 receptor is effective to ameliorate mucosal inflammation.

Thus, in one aspect of the invention, a method of treating mucosal inflammation associated with a pathological condition in a mammal is provided comprising the step of inhibiting 5-HT signaling at a target site, wherein 5-HT signaling is inhibited at the 5-HT7 receptor.

In another aspect of the invention, an article of manufacture is provided comprising packaging material and a composition, wherein the composition comprises an inhibitor of the 5-HT7 receptor, and the packaging material is labeled to indicate that the composition is for the treatment of mucosal inflammation.

These and other aspects of the invention will become apparent by in the detailed description by reference to the figures.

### BRIEF DESCRIPTION OF THE FIGURES

FIG. 1 graphically illustrates the disease activity index (A), macroscopic damage scores (B) and histological scores (C) in the development of DSS-induced colitis following 5-HT7 antagonist treatment;

FIG. 2 graphically illustrates the effect of 5-HT7 antagonist on MPO activity (A) and production of pro-inflammatory cytokines, IL-1β (B), TNF-α (C) and IL-6 (D);

FIG. 3 graphically illustrates the effects of the lack of the 5-HT7 receptor in the development of DSS-induced colitis as shown by the disease activity index (A), macroscopic damage scores (B) and histological scores (C);

FIG. 4 graphically illustrates the effect of lack of 5-HT7 receptor on MPO activity (A) and production of pro-inflammatory cytokines, IL-1β (B), TNF-α (C) and IL-6 (D) in DSS-induced colitis;

FIG. 5 graphically illustrates the effect of the lack of the 5-HT7 receptor on cytokine production by splenic dendritic cells;

FIG. 6 graphically illustrates the effects of lack of 5-HT7 on macroscopic damage scores (A) histologic damage scores (B), MPO activity (C) and IL-1β production (D) in DNBS-induced colitis;

FIG. 7 graphically illustrates the effects of a transfer of 5-HT7 deficient bone marrow cells on DAI (A), macroscopic damage (B), histological damage (C), MPO activity (D) and pro-inflammatory cytokines: IL-1β (E) TNF-α (F) and IL-6 (G) in DSS-induced colitis;

FIG. 8 graphically illustrates the effect of a transfer of 5-HT7 deficient bone marrow cells on cytokine production by splenic dendritic cells;

FIG. 9 graphically illustrates the effect of LPS and serotonin on IL-1β production by splenic dendritic cells after the transfer of 5-HT7 deficient bone marrow cells; and

FIG. 10 illustrates the gene (A) and amino acid (B) sequences of the 5-HT7a receptor.

### DETAILED DESCRIPTION OF THE INVENTION

A method of treating mucosal inflammation associated with a pathological condition in a mammal is provided comprising the step of inhibiting 5-HT signaling by blocking 5-HT7 receptor function at a target site.

Mucosal inflammation is used herein to refer to inflammation, e.g. a response to a harmful stimuli generally resulting in pain, swelling, redness, heat and/or loss of function, in the mucosa or mucous membrane, and particularly the mucosa of the gastrointestinal tract. Pathological conditions associated with inflammation in the gastrointestinal mucosa include, for example, colitis such as Inflammatory Bowel Disease, including ulcerative colitis and Crohn's disease, as well as infectious colitis.

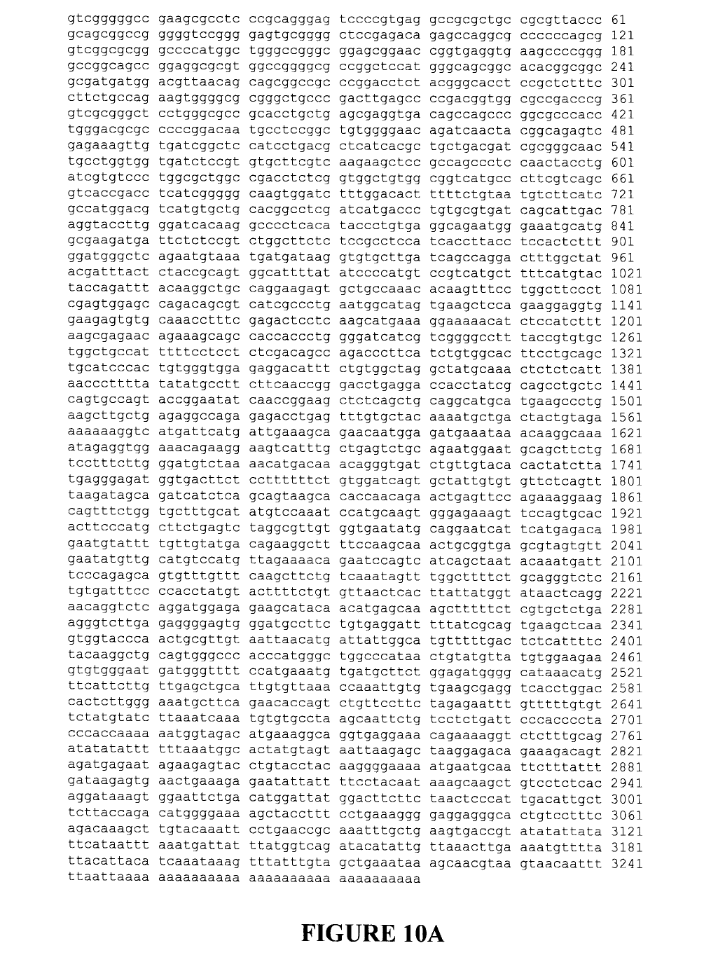

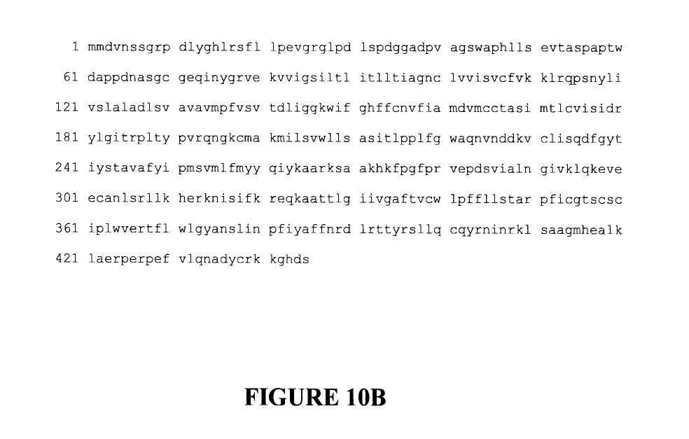

The term “5-HT7 receptor” refers to a cell surface G-protein coupled receptor that is activated by serotonin and encoded by an HTR7 gene. The term encompasses mammalian 5-HT7 receptors including both human and non-human receptors, and encompasses functionally equivalent 5-HT7 receptor variants, e.g. splice variants and different receptor isoforms. Human 5-HT7 receptor (5-HT₇₍ₐ₎) is a 445 amino acid protein, the sequence of which is illustrated in FIG. 10B. Examples of non-human 5-HT7 receptor include mouse (see RefSeq NP 032341), rat (see RefSeq NP 075227), and guinea pig (see RefSeq NP 001166435). Functionally equivalent splice variants of the 5-HT7 receptor, such as human 5-HT_{7(b)} and 5-HT_{7(d)} receptors that differ at their carboxy terminals. The 5-HT_{7(b)} receptor is a truncated 432 amino acid variant of 5-HT₇₍ₐ₎, while 5-HT_{7(d)} is a distinct 479 amino acid isoform in which an exon cassette is retained at the C-terminus. The term “functionally equivalent” refers to the function of the 5-HT7 receptor as a G-protein coupled receptor that is activated by serotonin.

The present method comprises inhibition of 5-HT signaling by blocking 5-HT7 receptor function. The term “inhibit”, “inhibiting” or “inhibition” is used herein to refer to any reduction of 5-HT signaling as a result of blockage or inhibition in connection with the 5-HT7 receptor, including both complete as well as partial reduction of 5-HT signaling. As one of skill in the art will appreciate, inhibition of 5-HT signaling by blockage of 5-HT7 receptor function may be achieved at the nucleic acid level, e.g. inhibition of nucleic levels or expression of the 5-HT7 receptor, or at the protein level, e.g. inhibition of 5-HT7 receptor function or activity. In either case, the result of blocking, e.g. inhibiting, or at least reducing, 5-HT7 receptor function is achieved. Inhibition of 5-HT signaling in accordance with the invention may be at a level sufficient to result in a reduction of mucosal inflammation, for example, a reduction in mucosal inflammation of at least about 10%, more preferably at least about 20%, 25%, 30%, or greater.

5-HT7 gene expression may be inhibited using well-established methodologies utilizing polynucleotides, such as anti-sense, snp or siRNA technologies, which are derived from 5-HT7-encoding nucleic acid molecules such as the sequence shown in FIG. 10A. Such a 5-HT7-encoding nucleic acid sequence, thus, may be used to prepare antisense oligonucleotides effective to bind to 5-HT7-encoding nucleic acid and inhibit the expression thereof. The term “antisense oligonucleotide” as used herein means a nucleotide sequence that is complementary to at least a portion of a target 5-HT7 nucleic acid sequence. The term “oligonucleotide” refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells) as well as the antisense binding region. In addition, two or more antisense oligonucleotides may be linked to form a chimeric oligonucleotide.

The antisense oligonucleotides of the present invention may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halo uracil, 5-halo cytosine, 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydrodyl guanine and other 8-substituted guanines, other aza and deaza uracils, thymidines, cytosines, adenines, or guanines, 5-tri-fluoromethyl uracil and 5-trifluoro cytosine.

Other antisense oligonucleotides of the invention may contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates and phosphorodithioates. In addition, the antisense oligonucleotides may contain a combination of linkages, for example, phosphorothioate bonds may link only the four to six 3′-terminal bases, may link all the nucleotides or may link only 1 pair of bases.

The antisense oligonucleotides of the invention may also comprise nucleotide analogs that may be better suited as therapeutic or experimental reagents. An example of an oligonucleotide analogue is a peptide nucleic acid (PNA) in which the deoxribose (or ribose) phosphate backbone in the DNA (or RNA), is replaced with a polymide backbone which is similar to that found in peptides (P. E. Nielson, et al Science 1991, 254, 1497). PNA analogues have been shown to be resistant to degradation by enzymes and to have extended lives in vivo and in vitro. PNAs also form stronger bonds with a complementary DNA sequence due to the lack of charge repulsion between the PNA strand and the DNA strand. Other oligonucleotide analogues may contain nucleotides having polymer backbones, cyclic backbones, or acyclic backbones. For example, the nucleotides may have morpholino backbone structures (U.S. Pat. No. 5,034,506). Oligonucleotide analogues may also contain groups such as reporter groups, protective groups and groups for improving the pharmacokinetic properties of the oligonucleotide. Antisense oligonucleotides may also incorporate sugar mimetics as will be appreciated by one of skill in the art.

Antisense nucleic acid molecules may be constructed using chemical synthesis and enzymatic ligation reactions using procedures known in the art based on a given 5-HT7 nucleic acid sequence such as that provided herein. The antisense nucleic acid molecules of the invention, or fragments thereof, may be chemically synthesized using naturally occurring nucleotides or variously modified nucleotides designed to increase the biological stability of the molecules or to increase the physical stability of the duplex formed with mRNA or the native gene, e.g. phosphorothioate derivatives and acridine substituted nucleotides. The antisense sequences may also be produced biologically. In this case, an antisense encoding nucleic acid is incorporated within an expression vector that is then introduced into cells in the form of a recombinant plasmid, phagemid or attenuated virus in which antisense sequences are produced under the control of a high efficiency regulatory region, the activity of which may be determined by the cell type into which the vector is introduced.

In another embodiment, siRNA technology may be applied to inhibit expression of 5-HT7. Application of nucleic acid fragments such as siRNA fragments that correspond with regions in a 5-HT7 gene and which selectively target a 5-HT7 gene may be used to block 5-HT7 expression. Such blocking occurs when the siRNA fragments bind to the gene thereby preventing translation of the gene to yield functional 5-HT7.

SiRNA, small interfering RNA molecules, corresponding to a region in the 5-HT7 gene are made using well-established methods of nucleic acid syntheses as outlined above with respect to antisense oligonucleotides. Since the structure of target 5-HT7 genes is known, fragments of RNA that correspond therewith can readily be made. The effectiveness of selected siRNA to block 5-HT7 expression can be confirmed using a 5-HT-7-expressing cell line. Briefly, selected siRNA may be incubated with a 5-HT7-expressing cell line under appropriate growth conditions. Following a sufficient reaction time, i.e. for the siRNA to bind with mRNA encoding 5-HT7 to result in decreased levels of free 5-HT7 mRNA, the reaction mixture is tested to determine if such a decrease has occurred. Suitable siRNA will prevent processing of the 5-117′7 gene to yield functional receptor. This can be detected by assaying for 5-HT7 activity in a cell-based assay, for example, to identify expression of a reporter gene that is regulated by 5-HT7 binding.

It will be appreciated by one of skill in the art that siRNA fragments useful in the present method may be derived from specific regions of 5-HT7-encoding nucleic acid which may provide more effective inhibition of gene expression, for example, at the 5′ end or the central region of the gene. In addition, as one of skill in the art will appreciate, useful siRNA fragments need not correspond exactly with a 5-HT7 target gene, but may incorporate sequence modifications, for example, addition, deletion or substitution of one or more of the nucleotide bases therein, provided that the modified siRNA retains the ability to bind selectively to the target gene. Selected siRNA fragments may additionally be modified in order to yield fragments that are more desirable for use. For example, siRNA fragments may be modified to attain increased stability in a manner similar to that described for antisense oligonucleotides.

Once prepared, oligonucleotides determined to be useful to inhibit 5-HT7 gene expression, such as antisense oligonucleotides and siRNA, may be used in a therapeutic method to treat mucosal inflammation in a mammal. A suitable oligonucleotide may be introduced into tissues or cells of the mammal using techniques in the art including vectors (retroviral vectors, adenoviral vectors and DNA virus vectors) or by using physical techniques such as microinjection.

Blockage of 5-HT7 receptor function may be inhibited at the protein level, for example, using inhibitors designed to block 5-HT7 activity either directly or indirectly. 5-HT7 inhibitors may include, for example, biological compounds, synthetic small molecules or peptide mimetics based on such biological compounds.

Examples of biological 5-HT7 inhibitors include immunological inhibitors such as polyclonal antibodies, or monoclonal antibodies prepared using well-established hybridoma technology developed by Kohler and Milstein (Nature 256, 495-497 (1975)). Hybridoma cells can be screened immunochemically for production of antibodies specifically reactive with a selected region of the 5-HT7 receptor and the monoclonal antibodies can be isolated. The term “antibody” as used herein is intended to include fragments thereof which also specifically react with a 5-HT7 receptor according to the invention, as well as chimeric antibody derivatives, i.e., antibody molecules resulting from the combination of a variable non-human animal peptide region and a constant human peptide region. Examples of 5-HT7 antibodies include LS-A7991, LS-A6673 and LS-C122418 which are commercially available from Lifespan Biosciences.

Candidate inhibitors of 5-HT7 receptor such as synthetic small molecules may also be employed to block 5-HT7 receptor function. In this regard, 5-HT7 receptor antagonists such as, but not limited to, 3-{4-[4-(4-chlorophenyl)-piperazin-1-yl]-butyl}-3-ethyl-6-fluoro-1,3-dihydro-2H-indol-2-one, Amisulpride, Amitriptyline, Amoxapine, Aripiprazole, Clomipramine, Clozapine, Cyproheptadine, N,N-Dimethyltryptamine, Fluphenazine, Fluperlapine, ICI-169,369 ((1R)-3,N-dimethyl-N-[1-methyl-3-(4-methylpiperidin-1-yl)propyl]benzenesulfonamide), Imipramine, Ketanserin, Loxapine, LSD, LY-215,840, Mesulergine, Mianserin, SB-258,719, SB-258,741, SB-269,970, SB-656,104-A, SB-691,673, Spiperone, Tenilapine and Zotepine may be employed in the present method. Although these antagonists may be readily synthesized using established methods of chemical synthesis, these antagonists are commercially available. As one of skill in the art will appreciate, prodrugs of any of such antagonists, or pharmaceutically acceptable salts, hydrates or solvates thereof, may also be employed. The term “prodrug” refers to a compound (e.g. a drug precursor) that is transformed in vivo to yield the inhibitor or a pharmaceutically acceptable analogue, salt, hydrate or solvate thereof. The transformation may occur by various mechanisms (e.g., by metabolic or chemical processes), such as, for example, through hydrolysis in blood. The term “salt(s)”, as employed herein, denotes both acidic salts formed with inorganic and/or organic acids, as well as basic salts formed with inorganic and/or organic bases. Pharmaceutically acceptable (i.e., non-toxic, physiologically acceptable) salts are preferred, although other salts are also useful. A “solvate” is formed by admixture of the inhibitor or an analogue thereof in a solvent which is preferably pharmaceutically acceptable.

Peptide mimetics may also be prepared, for example, based on known biological inhibitors, which block 5-HT7 receptor function. Such peptide mimetics may be designed to incorporate desirable features such as increased stability, e.g. resistant to biochemical degradation. Generally, such peptide mimetics are designed using techniques well-established in the art, including computer modeling, and prepared using standard methods of peptide synthesis.

Candidate inhibitors may be screened for inhibitory activity in a cell-based system. Suitable assays utilize primary or established 5-HT7-expressing cell lines, such as dentritic cell lines. 5-HT7 activity in the presence of a candidate compound may be monitored in such cell lines by measuring the level of one or more markers of activity including, but not limited to, mRNA or protein levels of 5-HT7, cyclic-adenosine monophosphate (cAMP) levels, MPO activity, cytokine levels (e.g. IL-12, IL-1β, TNF-α, IL-6) and other outputs such as protein activity, cell function, cell activities, and the like. In the presence of a compound which inhibits 5-HT7, cAMP levels will be reduced in comparison to control levels. As will be appreciated by one of skill in the art, the levels of markers of 5-HT7 inhibition may be determined using one or more of a number of standard techniques such as slot blots or western blots (for protein quantitation) or Q-PCR (for mRNA quantitation) in suitable cell culture following incubation with the candidate inhibitor for a suitable period of time, for example 24-48 hours.

A therapeutic inhibitor of 5-HT7 may be administered to a mammal to modulate 5-HT signaling in the treatment of mucosal inflammation. The inhibitor may be administered in combination with a suitable pharmaceutically acceptable carrier. The expression “pharmaceutically acceptable” means acceptable for use in the pharmaceutical and veterinary arts, i.e. not being unacceptably toxic or otherwise unsuitable. Examples of pharmaceutically acceptable carriers include diluents, excipients and the like. Reference may be made to “Remington's: The Science and Practice of Pharmacy”, 21st Ed., Lippincott Williams & Wilkins, 2005, for guidance on drug formulations generally. The selection of adjuvant depends on the type of inhibitor and the intended mode of administration of the composition. In one embodiment of the invention, the compounds are formulated for administration by infusion, or by injection either subcutaneously, intravenously, intrathecally, intraspinally or as part of an artificial matrix, and are accordingly utilized as aqueous solutions in sterile and pyrogen-free form and optionally buffered or made isotonic. Thus, a selected compound may be administered in distilled water or, more desirably, in saline, phosphate-buffered saline or 5% dextrose solution. Compositions for oral administration via tablet, capsule or suspension are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present. Other adjuvants may also be added to the composition regardless of how it is to be administered, for example, anti-microbial agents may be added to the composition to prevent microbial growth over prolonged storage periods.

A 5-HT7 inhibitor may be administered to a mammal in combination with other therapeutic agents to enhance the treatment mucosal inflammation. For example, a 5-HT7 inhibitor may be utilized in conjunction with conventional therapy for Inflammatory Bowel Disease, for example, in conjunction with an immunosuppressive drug, e.g. mesalazine (5-amino-2-hydroxybenzoic acid).

To treat mucosal inflammation in accordance with the present method, a therapeutically effective amount of 5-HT7 inhibition is attained by methods such as those described. The term “therapeutically effective” with respect to 5-HT7 inhibition is meant to refer to a level of inhibition that reduces 5-HT signaling to a level that functions to ameliorate inflammation of the mucosa. In this regard, 5-HT7 inhibition that results in a reduction of inflammation is therapeutically effective, e.g. a reduction in inflammation of at least about 10%, preferably at least about 20%, and more preferably at least about 25% or greater. The dosage of a 5-HT7 inhibitor that would be sufficient to achieve therapeutically effective 5-HT7 inhibition can readily be determined using appropriately controlled clinical trials, as one of skill in the art would appreciate. For synthetic small molecule inhibitors of 5-HT7, suitable dosages may also be determined based on current knowledge of these inhibitors. For example, it is expected that the therapeutically effective dosage of a 5-HT7 inhibitor such as SB-269,970, or a prodrug, salt, hydrate or solvate, thereof, would be in the range of about 0.1 to 1000 mg/kg, preferably a range of about 0.5-500 mg/kg, and more preferably a range of about 1-100 mg/kg.

In another aspect of the present invention, an article of manufacture is provided. The article comprises packaging material and a composition. The composition comprises a 5-HT7 inhibitor and a pharmaceutically acceptable carrier. The packaging material includes an indication that the composition is effective to treat a pathological condition involving mucosal inflammation. Examples of suitable 5-HT7 inhibitors are described above. Examples of pathological conditions involving intestinal mucosal inflammation include Inflammatory Bowel Disease and infectious colitis.

Embodiments of the invention are described in the following specific examples which are not to be construed as limiting.

### Example 1

### Materials and Methods

Animals.

C57BL/6 mice (Taconic) were kept in sterilized, filter-topped cages under specific pathogen-free conditions and fed autoclaved food. All mice were male aged 8-10 weeks. 5-HT7^{-/-} mice on C57BL/6 background were originally generated by a targeted gene disruption of the 5-HT7 receptor gene as described by Hedlund et al. (Proc Natl Acad Sci USA 2003 Feb. 4; 100(3):1375-80). These mice were viable and showed no observed difference in food intake or body weight compared to wild type mice. Breeding pairs were obtained from Peter B. Hedlund (The Scripps Research Institute, La Jolla, Calif., USA) and were kept and bred under specific pathogen free conditions. All experiments were approved by the animal ethics committee of McMaster University and conducted under the Canadian guidelines for animal research.

Drugs.

Mice were treated with selective 5-HT₇ antagonist SB-269970 ((2R)-1-[(3-Hydroxyphenyl)sulfonyl]-2-[2-(4-methyl-1-piperidinyl)ethyl]pyrrolidinehydrochloride) purchased from ToCris Biosciences (Burlington, ON, Canada) and dissolved in distilled water. The antagonist was administered intraperitoneally at a dosage of 40 mg/kg. Control mice received saline as vehicle.

Induction of DSS and DNBS Colitis.

Dextran sulfate sodium (DSS) (MW 40 kDa; ICN, Biomedicals Incorporate, Solon, Ohio, USA) was added to drinking water for a final concentration of 5% (wt/volume) for a total of 5 days. Mean DSS consumption was noted per cage each day. For DNBS induced colitis, mice were anesthetized with isoflurane (Abbott, Toronto, Canada). A 10-cm long tubing attached to a tuberculin syringe was intrarectally inserted 3.5 cm into the colon and in order to induce colitis, 100 μL of 5 mg of DNBS solution (ICN, Biomedicals Inc) dissolved in 50% Ethanol was administered and left for 3 days. Controls received only 50% Ethanol for the same time span. Mice in which colitis was induced were supplied with 6% sucrose in their drinking water in order to prevent dehydration.

Experimental Protocol.

C57BL/6 (5-HT7^{+/+}) and 5-HT7^{-/-} mice were exposed to 5% DSS for 5 days. In a separate experiment, C57BL/6 mice were treated with SB-269970 (at a dosage of 40 mg/kg) or vehicle (saline) intraperitoneally for 6 days starting one day prior to exposure to DSS. For DNBS experimental colitis, DNBS (5 mg) solution was administered and left for 3 days; control mice received 50% Ethanol only. During DSS administration, the disease activity index is used to assess the onset of colitis (SI Text). To assess macroscopic damage, mice were sacrificed 5 days post-DSS or 3 days post-DNBS administration. Colonic tissue samples were collected for histological analysis and to evaluate myeloperoxidase activity, serotonin levels, and pro-inflammatory cytokine levels.

Assessment of Onset of Colitis.

Disease Activity Index (DAI) is a combined score of weight loss, stool consistency, and fecal bleeding. This scoring system was defined as: weight loss: 0, no loss; 1, 1-5%; 2, 5-10%; 3, 10-20%; 4, 20%+; stool: 0, normal; 2, loose stool; 4, diarrhea; and bleeding: 0, no blood, 2, Hemoccult positive (Hemoccult II, Beckman Coulter, Fullerton, Calif.); and 4, gross blood (blood around anus). DAI was measured on all 5 days of DSS treatment.

Assessment of Severity of Colitis.

For assessing macroscopic damage, after 5 days from the beginning of DSS or 3 days from the beginning of DNBS treatment, mice were sacrificed, the abdominal cavity was opened, and observations on colonic distension, fluid content, hyperemia, and erythema were recorded. The colon was removed and macroscopic damage was immediately assessed on the full section of the colon. Macroscopic scores were performed using a previously described scoring system for DSS colitis (Cooper et al. Lab Invest 1993 August; 69(2):238-49) and for DNBS (Khan et al. 2002. Infect Immun 70:5931-5937).

Colonic Histology and MPO Activity.

Formalin-fixed colon segments were paraffin-embedded and 3-μm sections were stained with hematoxylin and eosin. Colonic damage was blindly scored based on the DSS colitis scoring system noted above. This scoring system considers loss of architecture (0, normal-3, severe), cellular infiltration (0, normal-3, severe), muscle thickening (0, normal-3, severe), goblet cell depletion (0, absent; 1, present), crypt abscess (0, absent; 1, present). MPO (myeloperoxidase) is an enzyme contained in granulocytes such as neutrophils and is used as an index of inflammation. MPO activity was measured using a previously published protocol (Khan et al. 2002, Ibid). Briefly, colonic tissue samples were homogenized in ice-cold 50 mmol/L potassium phosphate buffer (pH=6.0) containing 0.5% hexadecyl trimethyl ammonium bromide (Sigma). Homogenates were centrifuged for 6 min (13,400×g, 4° C.). The supernatant was removed and an aliquot (7 μL) was then added to a solution containing potassium phosphate buffer, O-dianisidine (Sigma) and hydrogen peroxide. The absorbance was measured at 450 nm by a spectrophotometer (BioTek, model EL808). MPO activity was expressed in units per milligram of wet tissue, where 1 unit is the quantity of enzyme able to convert 1 mmol of hydrogen peroxide to water in 1 minute at room temperature.

Colonic Tissue Cytokine Levels.

Colonic samples were homogenized in 1 mL of Tris.HCl buffer containing protein inhibitors (Sigma). Samples were then centrifuged and the supernatant was frozen at −80° C. until the assay was conducted. Cytokine levels (IL-1β, TNF-α, IL-6) were determined using a commercially available enzyme-linked immunosorbent assay kit (Quantikine Murine; R&D Systems, Minneapolis, Minn., USA).

Isolation of DCs from Spleens.

Mice were sacrificed by cervical dislocation and spleens were excised and placed in Spleen Dissociation Medium (STEMCELL Technologies) and incubated for 30 min at room temperature. They were then strained through a 70-μm nylon mesh filter (BD Falcon) and washed with PBS supplemented with 2% fetal bovine serum (FBS) and 1 mmol/L EDTA. Splenic DCs were isolated using a CD11c⁺ isolation kit (EasySep®, STEMCELL Technologies) according to the manufacturer's guidelines.

Ex Vivo DC Culture.

DCs isolated using CD11c positive selection were incubated at 1×10⁶ cells per mL for 24 hours at 37° C. with or without 100 ng/mL LPS (Sigma-Aldrich) in RPMI 1640 supplemented with 10% FBS, 100 U/mL penicillin, 100 μg/mL streptomycin, and 50 μM 2-mercaptoethanol (Invitrogen Life Technologies). Supernatents were collected after 24 hours and analyzed for cytokine levels using ELISA kits for murine IL-12p40, IL-1β, and IL-6 (Quantikine Murine; R&D Systems, Minneapolis, Minn., USA).

Irradiation and Bone Marrow Transplantation.

6-8 week old male recipient C57BL/6 mice (Taconic) were irradiated with two doses of 5.5 Gy 48 hours apart administered via a ¹³⁷Cs γ-irradiation source (Gamma Cell 40; Nordian, Kanata, ON, Canada). Bone marrow cells (no fewer than 5×10⁴) were harvested from femurs and tibiae of donor mice (5-HT7^{+/+}) and 5-HT7^{-/-} and given via tail vein injection to recipient mice within two hours of the second irradiation exposure. Bone marrow cells were depleted of T-cells by incubation with a cocktail of in-house anti-CD4 (GK1.5), anti-CD8 (2.43), and anti-Thy 1.2 (supplied by Dr. Jonathan Bramson, McMaster University) at 4° C. for 1 hour followed by treatment with Low-Tox-M Guinea pig complement (Cedarlane) for 1 hour at 37° C. Recipient mice received antibiotics (Novo-Trimel®, Novopharm) starting 3-4 prior to first radiation exposure and for 3-4 weeks post-engraftment.

Antibodies.

Splenocyte preparations were surface stained with various monoclonal antibodies and used for flow cytometry assays. The antibodies used were: anti-CD11b (clone M1/70), anti-CD11c (clone HL3), anti-CD80-PerCP-Cy5.5, anti-CD86 (clone GL1), anti-MHC II (clone 25-9-17), and anti-CD40 (clone 3/23). All antibodies were purchased from BD Biosciences. Data were acquired using a FACSCanto flow cytometer with FACSDiva 5.0.2 software (BD Pharmingen) and analyzed with FlowJo Mac, version 6.3.4 software (Treestar, Ashland, Oreg.).

Statistical Analysis of Data.

Statistical analysis was performed using GraphPad Prism version 5.04 software (GraphPad Software, San Diego, Calif., USA) using a one way ANOVA followed by Student-Newman-Keuls multiple comparisons post hoc analysis. All data are presented as means±SEM and values of P<0.05 were considered significant.

### Results

5-HT7 Antagonist Delays Onset and Decreases the Severity of DSS-Induced Colitis

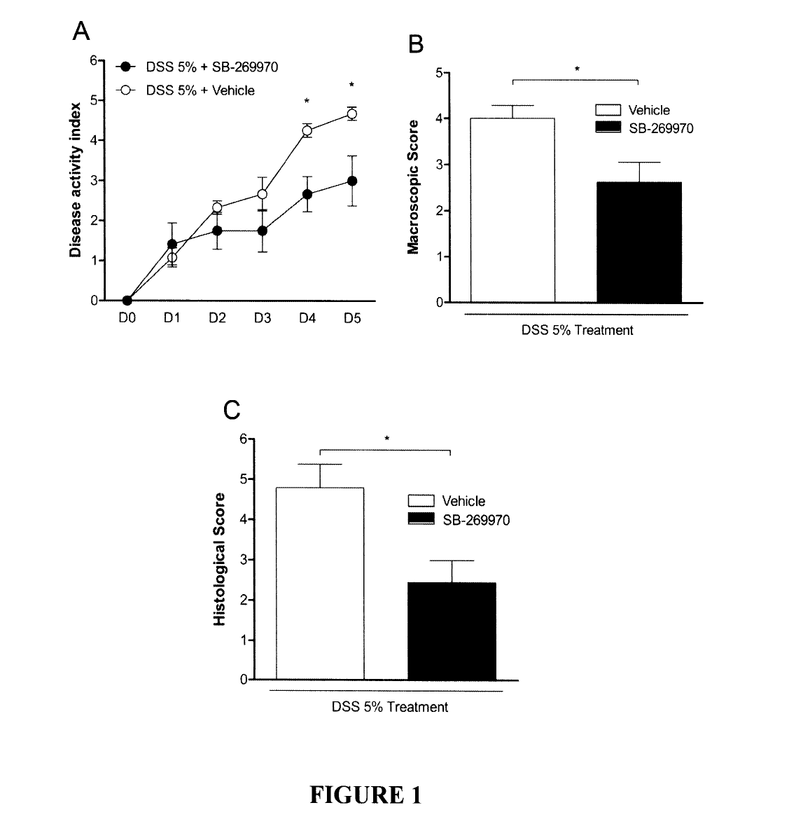

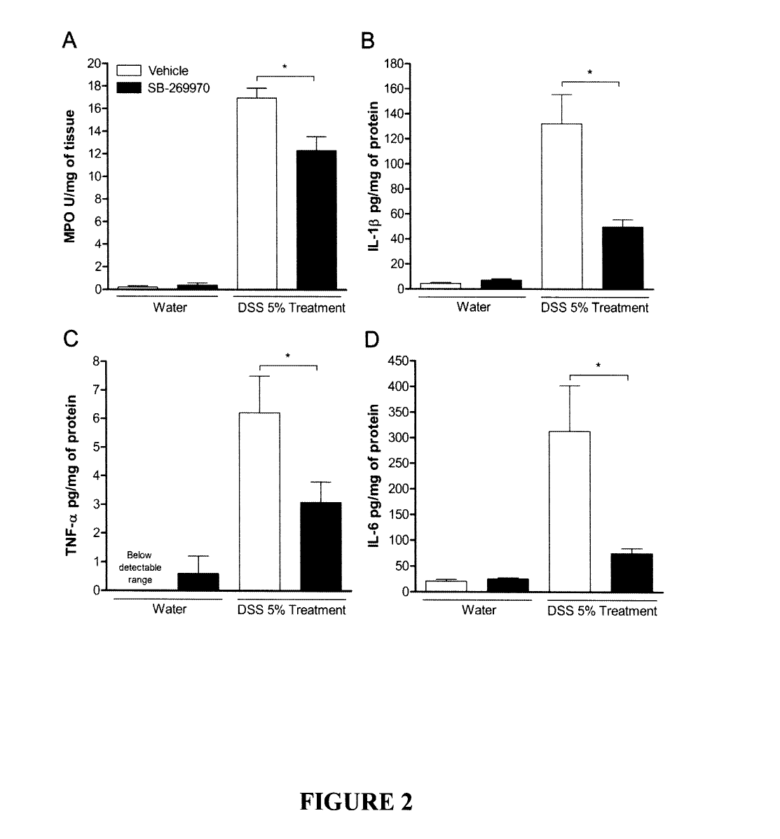

Clinical disease activity scores (fecal blood and consistency, and weight loss) were significantly lower in mice that were treated with the 5-HT7 antagonist (SB-269970, 40 mg/kg) on days 4 and 5 post-DSS administration (FIG. 1A). In vehicle (saline) treated mice, exposure to DSS in drinking water induced colitis as characterized by rectal bleeding, fecal bleeding, diarrhea, and weight loss. H&E stained colonic tissue sections showed increased leukocyte infiltration, loss of goblet cells, and distortion of epithelial cell architecture as well as thickening of the muscularis mucosa layer (FIG. 1B). In mice that received SB-269970, colitis severity was significantly lower and histological scores were significantly less severe compared to controls on day 5 post-DSS induction (FIG. 1C). This decrease in colitis severity was associated with significantly lower myeloperoxidase (MPO) activity (FIG. 2A) and lower production of pro-inflammatory cytokines including IL-1β (FIG. 2B), TNF-α (FIG. 2C), and IL-6 (FIG. 2D) in colonic tissue.

Targeted Disruption of 5-HT7 Decreases the Severity of DSS-Induced Colitis

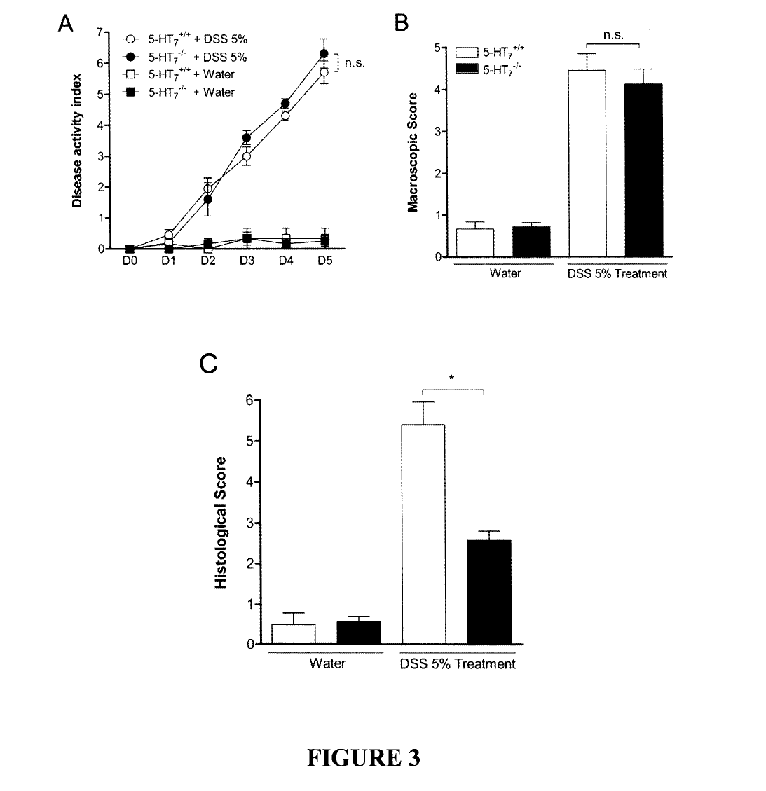

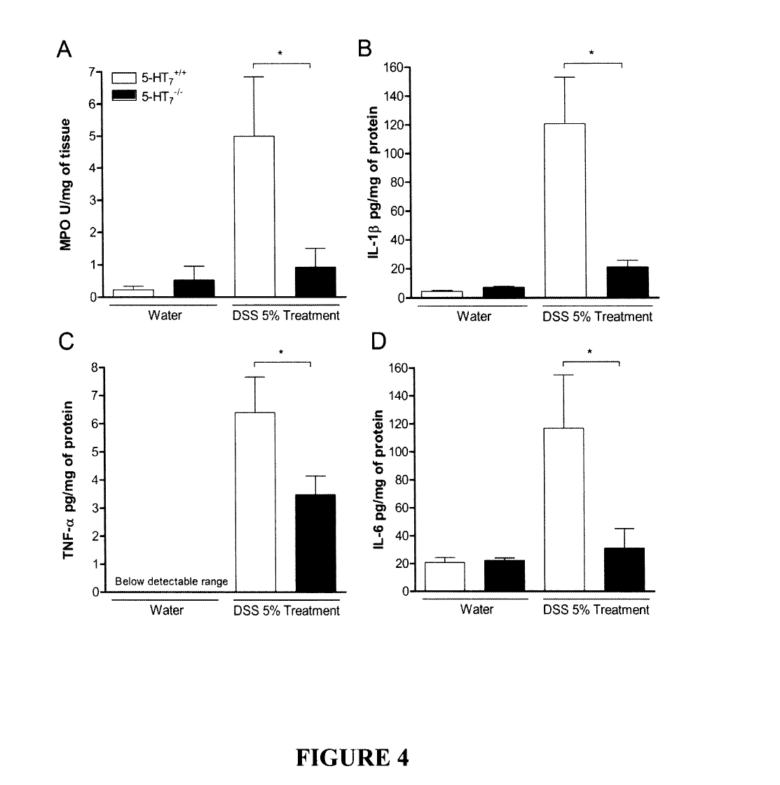

While disease onset and macroscopic scores were not significantly different between wild-type (5-HT7^{+/+}) and 5-HT7^{-/-} mice (FIG. 3A/B), histological scores were significantly less severe (FIG. 3C) and MPO activity and pro-inflammatory cytokine levels were significantly lower in 5-HT7^{-/-} as compared to wild-type controls (FIG. 4). There were no significant differences in colonic 5-HT levels between the groups (5-HT amount was 4.03±0.26 ng/mg of tissue and 4.57±0.31 ng/mg of tissue in wild-type (5-HT7^{+/+}) and 5-HT7^{-/-} mice following DSS induction, respectively). There was no difference in food intake between groups (daily average food intake was 3.16±0.19 g/mouse and 3.48±0.25 g/mouse in wild-type and 5-HT7^{-/-} mice post-DSS induction, respectively).

Down-Regulation of Cytokine Production in DCs with Disrupted 5-HT7 Function.

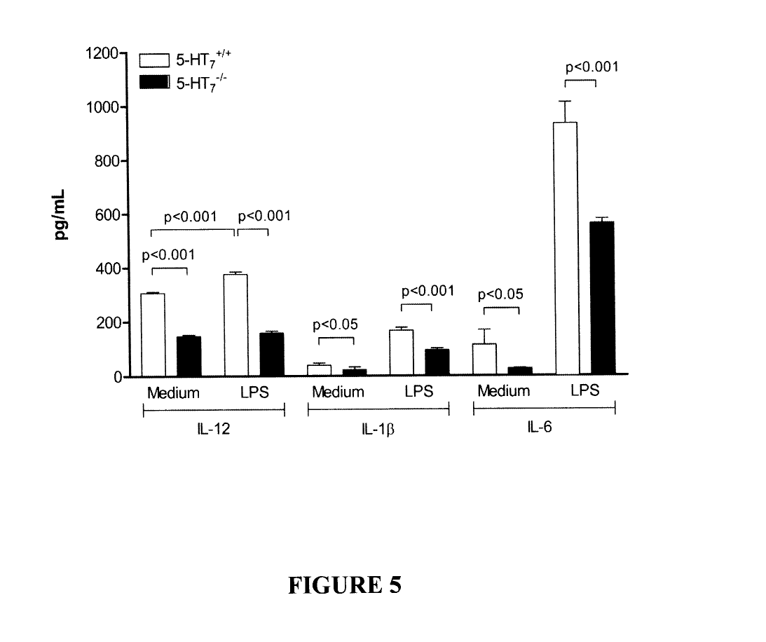

In order to investigate whether 5-HT can mediate DC cytokine production in gut inflammations by acting through the 5-HT7 receptor, cytokine production was assessed using culture supernatants of DCs isolated from DSS-treated wild-type (5-HT7^{+/+}) and 5-HT7^{-/-} mice with or without LPS. DCs isolated from 5-HT7^{-/-} mice post-DSS produced lower levels of IL-12, IL-1β, and IL-6 when stimulated with LPS as compared to wild-type mice (FIG. 5).

Targeted Disruption of 5-HT₇ Decreases the Severity of DNBS-Induced Colitis

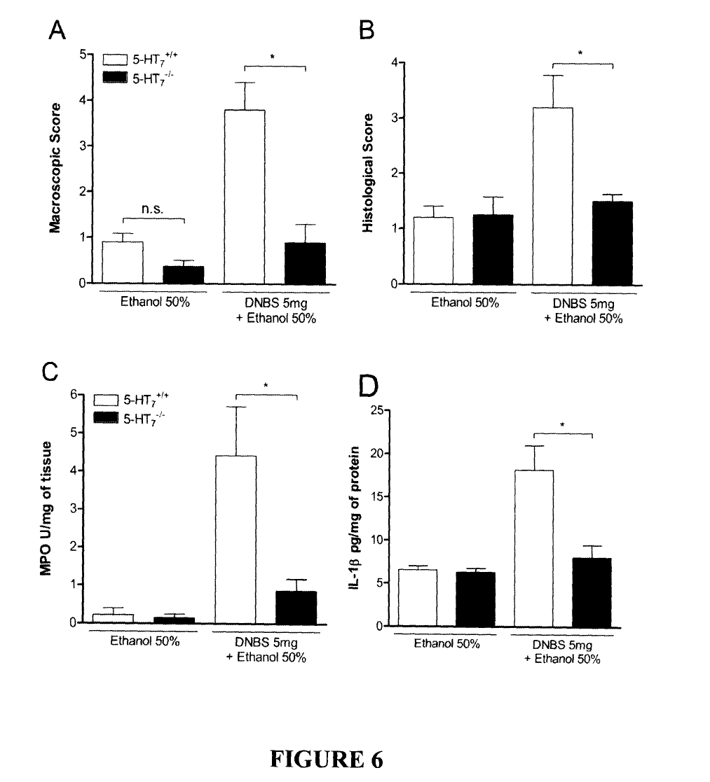

To determine whether the aforementioned changes were specific only to the DSS model of colitis, another model of experimental colitis (the DNBS-based model) was utilized in wild-type (5-HT7^{+/+}) and 5-HT7^{-/-} mice. In wild-type mice, DNBS exposure caused significant thickening of the colonic wall, hyperemia, observable adhesion between the colon and surrounding tissue, and in some cases, ulcerations. H&E stained colonic tissue sections of wild-type mice given DNBS showed increased cellular infiltration, loss of goblet cells, and severe mucosal damage. 5-HT7^{-/-} mice had significantly reduced colitis severity (FIG. 6A) and less severe histological scores (less mucosal damage, less cellular infiltration and goblet cell depletion) (FIG. 6B). Reduction in severity of colitis in 5-HT7^{-/-} mice treated with DNBS was associated with reduced MPO activity (FIG. 6C) and lower colonic IL-1β levels (FIG. 6D) compared to controls.

Reconstitution with 5-HT7 Deficient Bone Marrow Results in Reduced Colitis Severity

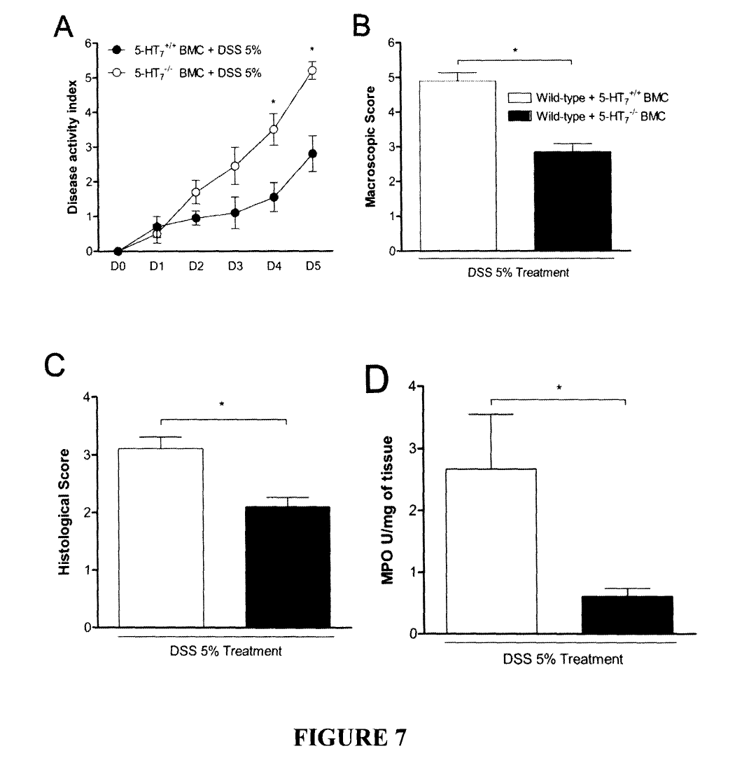

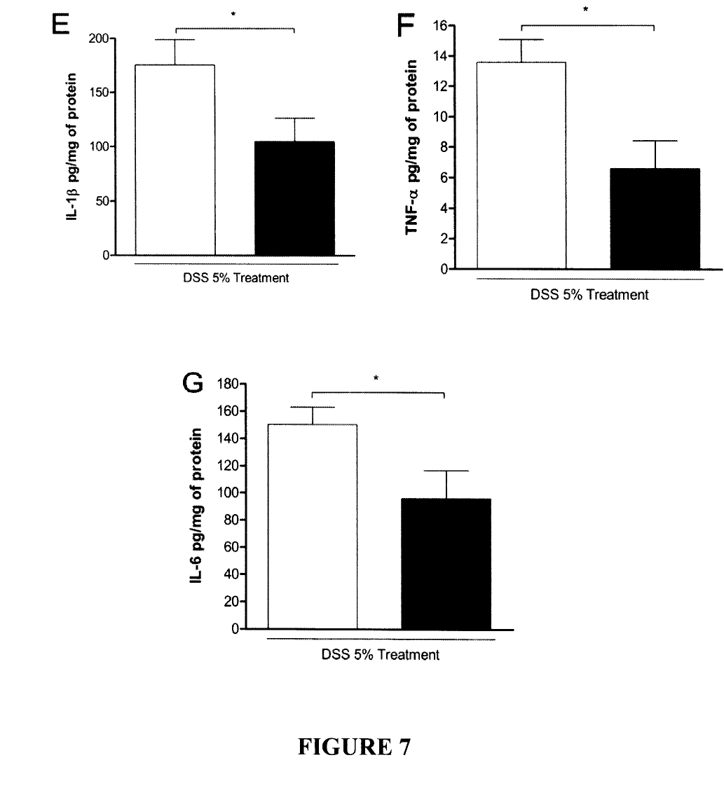

To further confirm the role of the 5-HT7 receptor in immune cell activation and in generation of inflammation, lethally irradiated wild-type (5-HT7^{+/+}) mice were reconstituted with bone marrow cells (BMC) harvested from wild-type (5-HT7^{+/+}) or 5-HT7^{-/-} mice via tail vein injections. Lymphocyte depletion in bone marrow cell preparations were confirmed prior to injections by flow cytometry. Reconstituted mice were given 5% DSS ad libitum for 5 days. Mice reconstituted with BMC harvested from 5-HT7^{-/-} mice had lower DAI scores (less weight loss, fecal blood and consistency) on days 4 and 5 of DSS exposure as compared to control mice that were reconstituted with bone marrow cells harvested from wild-type (5-HT7^{+/+}) mice (FIG. 7A). Disease severity and histological scores were significantly lower in the transgenic mice post-DSS (FIGS. 7B/C) and this was associated with reduced MPO activity (FIG. 7D) and lower production of pro-inflammatory cytokines including IL-1β (FIG. 7E), TNF-α (FIG. 7F) and IL-6 (FIG. 7G). The significant reduction of 5-HT7 expression in transgenic mice was verified by leukocyte RNA extraction and analysis by real-time PCR (data not shown).

Altered Cytokine Production from DCs Isolated from Radiation-Induced Chimeric Mice after Reconstitution

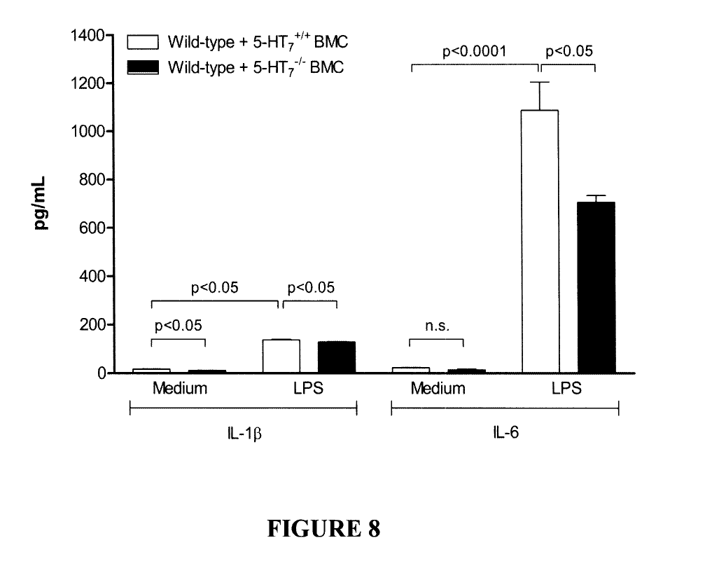

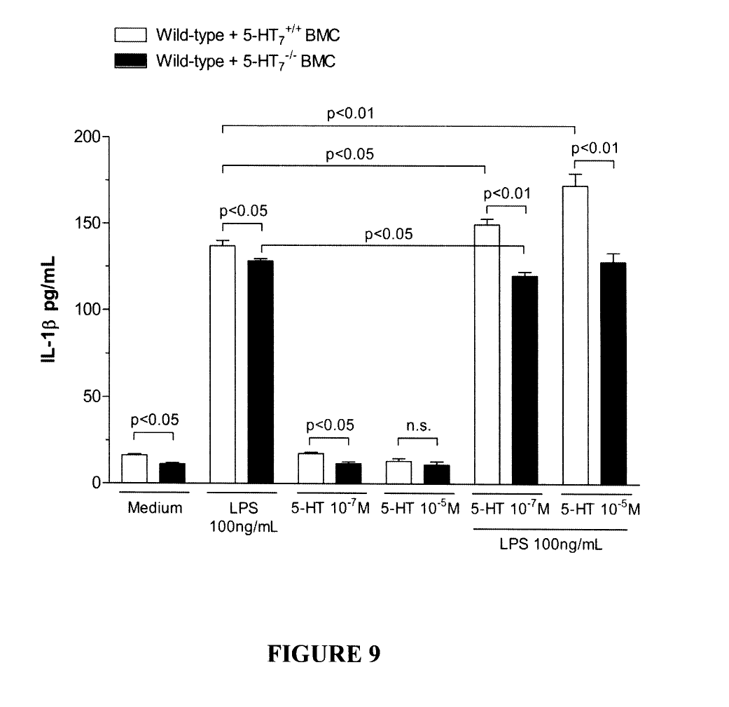

CD11c positive DCs were isolated from spleens of irradiated mice reconstituted with BMC from 5-HT7^{+/+} and 5-HT7^{+/+} mice post-DSS administration. DCs isolated from mice given BMCs from 5-HT7^{-/-} produced significantly lower levels of IL-1β and IL-6 in the presence of LPS when compared to DCs isolated from controls (FIG. 8). The presence of both LPS and serotonin in the culture media of CD11c positive DCs isolated from mice reconstituted with wild-type BMC significantly up-regulated IL-1β production compared to DCs cultured in the presence of LPS only (FIG. 9). This increase in IL-1β production was not seen in cultured DCs isolated from spleens of transgenic mice. To assess the phenotype of the respective cell populations, CD11c positive splenocytes from both experimental groups were stained for various DC markers and analyzed by flow cytometry. No significant difference in the expression levels of MHC class II molecules, CD40, or co-stimulatory molecules CD80 and CD86 were detected.

### Discussion

In summary, the data presented in this study show that the 5-HT7 receptor plays a critical role in regulation of mucosal inflammation and immune responses and that targeting the 5-HT7 receptor on DCs serves as a therapeutic strategy to ameliorate mucosal inflammation and intervene in inflammatory disorders such as IBD.

The relevant contents of all references referred to herein are incorporated by reference.

We claim:

1. A method of treating mucosal inflammation in the gastrointestinal tract associated with a pathological condition in a mammal comprising the step of inhibiting 5-HT signaling at a target site, wherein 5-HT signaling is inhibited at the 5-HT7 receptor.

2. The method of claim 1, wherein 5-HT signaling is inhibited by administration of a 5-HT7 receptor antagonist.

3. The method of claim 1, wherein the condition is colitis.

4. The method of claim 3, wherein the condition is inflammatory bowel disease.

5. The method of claim 2, wherein the receptor antagonist is selected from the group consisting of: 3-{4-[4-(4-chlorophenyl)-piperazin-1-yl]-butyl}-3-ethyl-6-fluoro-1,3-dihydro-2H-indol-2-one, Amisulpride, Amitriptyline, Amoxapine, Aripiprazole, Clomipramine, Clozapine, Cyproheptadine, N,N-Dimethyltryptamine, Fluphenazine, Fluperlapine, ICI-169,369 ((1R)-3,N-dimethyl-N-[1-methyl-3-(4-methylpiperidin-1-yl)propyl]benzenesulfonamide), Imipramine, Ketanserin, Loxapine, LSD, LY-215,840, Mesulergine, Mianserin, SB-258,719, SB-258,741, SB-269,970, SB-656,104-A, SB-691,673, Spiperone, Tenilapine, Zotepine, and pharmaceutically acceptable prodrugs, salts, solvates and hydrates thereof.

6. The method of claim 2, wherein the receptor antagonist is ((2R)-1-[(3-Hydroxyphenyl)sulfonyl]-2-[2-(4-methyl-1-piperidinyl)ethyl]pyrrolidinehydrochloride) or a pharmaceutically acceptable prodrug, salt, solvate or hydrate thereof.

7. The method of claim 1, wherein 5-HT signaling is inhibited by at least about 10%.

8. The method of claim 2, wherein the inhibitor is administered interperitoneally.

9. The method of claim 2, wherein the dosage of the inhibitor is at least about 1-100 mg/kg.

10. An article of manufacture comprising packaging material and a composition, wherein the composition comprises an inhibitor of the 5-HT7 receptor, and the packaging material is labeled to indicate that the composition is for the treatment of a pathological condition associated with mucosal inflammation in the intestinal tract of a mammal.

11. The article of claim 10, wherein the condition is colitis.

12. The article of claim 10, wherein the condition is inflammatory bowel disease.

13. The article of claim 10, wherein the inhibitor is selected from the group consisting of: 3-{4-[4-(4-chlorophenyl)-piperazin-1-yl]-butyl}-3-ethyl-6-fluoro-1,3-dihydro-2H-indol-2-one, Amisulpride, Amitriptyline, Amoxapine, Aripiprazole, Clomipramine, Clozapine, Cyproheptadine, N,N-Dimethyltryptamine, Fluphenazine, Fluperlapine, ICI-169,369 ((1R)-3,N-dimethyl-N-[1-methyl-3-(4-methylpiperidin-1-yl)propyl]benzenesulfonamide), Imipramine, Ketanserin, Loxapine, LSD, LY-215,840, Mesulergine, Mianserin, SB-258,719, SB-258,741, SB-269,970, SB-656,104-A, SB-691,673, Spiperone, Tenilapine, Zotepine and pharmaceutically acceptable prodrugs, salts, solvates and hydrates thereof.

14. The article of claim 10, wherein the inhibitor is receptor antagonist is ((2R)-1-[(3-Hydroxyphenyl)sulfonyl]-2-[2-(4-methyl-1-piperidinyl)ethyl]pyrrolidinehydrochloride) or a pharmaceutically acceptable prodrug, salt, solvate or hydrate thereof.

15. The article of claim 10, wherein the composition is suitable for injection into the mammal.
